SEQUENCE LISTING (10 sequences)

SEQ ID NO: 1 - protein, 498 aa, Homo sapiens
MTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 2 - protein, 503 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 3 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 4 - protein, 728 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 5 - protein, 574 aa, Homo sapiens
MCPRAARAPATLLLALGAVLWPAAGAWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHTDEMFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKVIYPAVEGRIKFSTGSHCHGSFSLIFLSLWAVIFVLYQ

SEQ ID NO: 6 - protein, 161 aa, Homo sapiens
MNLAISIALLLTVLQVSRGQKVTSLTACLVDQSLRLDCRHENTSSSPIQYEFSLTRETKKHVLFGTVGVPEHTYRSRTNFTSKYNMKVLYLSAFTSKDEGTYTCALHHSGHSPPISSQNVTVLRDKLVKCEGISLLAQNTSWLLLLLLSLSLLQATDFMSL

SEQ ID NO: 7 - protein, 658 aa, Homo sapiens
MDRGTLPLAVALLLASCSLSPTSLAETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAHLKCTITGLTFWDPSCEAEDRGDKFVLRSAYSSCGMQVSASMISNEAVVNILSSSSPQRKKVHCLNMDSLSFQLGLYLSPHFLQASNTIEPGQQSFVQVRVSPSVSEFLLQLDSCHLDLGPEGGTVELIQGRAAKGNCVSLLSPSPEGDPRFSFLLHFYTVPIPKTGTLSCTVALRPKTGSQDQEVHRTVFMRLNIISPDLSGCTSKGLVLPAVLGITFGAFLIGALLTAALWYIYSHTRSPSKREPVVAVAAPASSESSSTNHSIGSTQSTPCSTSSMA

SEQ ID NO: 8 - protein, 385 aa, Homo sapiens
MLVRRGARAGPRMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLGEDPYYTENGGGQGYSSGPGTSPEAQGKASVNRGAQENGTGQATSRNGHSARQHVVADTEL

SEQ ID NO: 9 - protein, 1304 aa, Homo sapiens
MYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 10 - protein, 254 aa, Homo sapiens
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGVRKSNAAERRGPL

(19) United States
(12) Patent Application Publication
(10) Pub. No.: US 2026/0185978 A1
(43) Pub. Date: Jul. 2, 2026

(54) METHOD OF ASSESSING WOUND HEALING POTENCY OF A MESENCHYMAL STEM POPULATION AND RELATED METHODS OF SELECTING MESENCHYMAL STEM CELLS AND IDENTIFYING TISSUE AS STARTING MATERIAL FOR PRODUCING A MESENCHYMAL STEM CELL POPULATION

(71) Applicant: CELLRESEARCH CORPORATION PTE. LTD., SINGAPORE (SG)

(72) Inventor: Toan Thang PHAN, Singapore (SG)

(73) Assignee: CELLRESEARCH CORPORATION PTE. LTD., SINGAPORE (SG)

(21) Appl. No.: 19/330,657

(22) Filed: Sep. 16, 2025

Related U.S. Application Data
(62) Division of application No. 17/066,378, filed on Oct. 8, 2020, now Pat. No. 12,449,413.
(60) Provisional application No. 62/912,374, filed on Oct. 8, 2019.

(51) Int. Cl. G01N 33/50; C12N 5/00; G01N 33/68

(52) U.S. Cl. CPC G01N 33/5005; C12N 5/0018; G01N 33/6803; G01N 2333/475; G01N 2333/4753; G01N 2333/495; G01N 2496/00

(57) ABSTRACT

The present invention relates to a method of assessing the wound healing potency of a mesenchymal stem cell population. In addition, the present invention concerns a method of selecting a mesenchymal stem cell population for producing a stem cell population under cGMP conditions and a method of selecting a mesenchymal stem cell population for producing a stem cell population for subsequent pharmaceutical administration. Further, the present invention relates to a method of selecting a mesenchymal stem cell population for generating a master cell bank and to a method of identifying a tissue suitable as starting material for producing a mesenchymal stem cell population for pharmaceutical use.

### CROSS-REFERENCE TO RELATED APPLICATIONS

The present application is a divisional of U.S. patent application Ser. No. 17/066,378, filed Oct. 8, 2020, which claims the benefit of priority of U.S. Provisional Application No. 62/912,374 filed Oct. 8, 2019, the contents of each of which are hereby incorporated by reference in their entirety for all purposes.

### SEQUENCE LISTING

The application contains a Sequence Listing which has been submitted electronically in .XML format and is hereby incorporated by reference in its entirety. Said .XML copy, created on Oct. 14, 2025, is named “SCH-5600-DV1.xml” and is 15,086 bytes in size. The sequence listing contained in this .XML file is part of the specification and is hereby incorporated by reference herein in its entirety.

### FIELD OF THE INVENTION

The present invention relates to a method of assessing the wound healing potency of a mesenchymal stem cell population. In addition, the present invention concerns a method of selecting a mesenchymal stem cell population for producing a stem cell population under cGMP conditions and a method of selecting a mesenchymal stem cell population for producing a stem cell population for subsequent pharmaceutical administration. Further, the present invention relates to a method of selecting a mesenchymal stem cell population for generating a master cell bank and to a method of identifying a tissue suitable as starting material for producing a mesenchymal stem cell population for pharmaceutical use. The present invention also concerns use of at least one protein for assessing the wound healing potency of a mesenchymal stem cell population. The present invention also relates to use of at least one protein for selecting a mesenchymal stem cell population for producing a stem cell population under cGMP conditions. Furthermore, the present invention relates to use of at least one protein for selecting a mesenchymal stem cell population for selecting a mesenchymal stem cell population for producing a stem cell population for subsequent pharmaceutical administration. The present invention also relates to use of at least one protein for selecting a mesenchymal stem cell population for selecting a mesenchymal stem cell population for generating a master cell bank and to a use of at least one protein for selecting a mesenchymal stem cell population for selecting a mesenchymal stem cell population for identifying a tissue suitable as starting material for producing a mesenchymal stem cell population for pharmaceutical use. Moreover, the present invention concerns a method of identifying a medium suitable for inducing or improving wound healing properties of a mesenchymal stem cell population.

### BACKGROUND OF THE INVENTION

Mesenchymal stem cells (MSCs) are capable of self-renewal and multilineage differentiation. Thereby, these cells are an attractive and promising tool for regenerative medicine. MSCs can be isolated from various tissues such as bone marrow stroma, fat tissue, dermis, placenta, umbilical cord blood or the different umbilical cord tissues comprising the Wharton's jelly, subendothelial layer of the umbilical cord vein, or the amniotic tissue of the umbilical cord (Mitchell, K. E. et al. (2003) Stem Cells 21, 50-60; U.S. Pat. No. 5,919,702; US Patent Application 2004/0136967; Romanov, Y. A. et al. (2003) Stem Cells 21, 105-110; Covas, D. T. et al. (2003) Braz. J. Med. Biol. Res. 36, 1179-1183; US2006/0078993). Mesenchymal stem cells isolated from the amniotic membrane of the umbilical cord have been first reported in US patent application 2006/0078993 (leading to granted U.S. Pat. Nos. 9,085,755, 9,737,568 and 9,844,571) and the corresponding International patent application WO2006/019357. In addition, a population of such mesenchymal stem cells from the amniotic membrane of the umbilical cord has recently been described in US application 20181/27721 or the corresponding International Application WO 2018/067071.

The mesenchymal stem cell population described in the US application 20181/27721 or the corresponding International Application WO 2018/067071 has the advantage that 99% or more of the stem cells of this population express the three MSC markers CD73, CD90 while lacking expression of CD34, CD45 and HLA-DR. This extremely homogenous and well defined cell population is thus an ideal candidate for clinical trials and cell based therapies as it, for example, fully meets the criteria generally accepted for human MSCs to be used for cellular therapy as defined, for example, by Dominici et al, “Minimal criteria for defining multipotent mesenchymal stromal cells. The International Society for Cellular Therapy position statement”, Cytotherapy (2006) Vol. 8, No. 4, 315-317, Sensebe et al. “Production of mesenchymal stromal/stem cells according to good manufacturing practices: a, review”, Stem Cell Research & Therapy 2013, 4:66), Vonk et al., Stem Cell Research & Therapy (2015) 6:94, or Kundrotas Acta Medica Lituanica. 2012. Vol. 19. No. 2. P. 75-79. As described in International Application WO 2018/067071, this mesenchymal stem cell population may, for example, be used in its undifferentiated state for wound healing purposes such as treatment of burns or chronic diabetic wounds. Alternatively, this mesenchymal stem cell population may be differentiated, for example, into insulin producing β-islet cells which can then be administered, for example by implantation, to a patient that suffers from an insulin deficiency such as diabetes mellitus (cf. also International Application WO2007/046775 in this respect).

The process of producing this mesenchymal stem cell population as also described in International Application WO 2018/067071 is suitable for being carried out under Good Manufacturing Practice (GMP) conditions, as required for such allogenic cell-based therapies. However, GMP production requires quality control of the manufactured drug product, regardless of whether the drug product is a small organic molecule, a biological molecule or even a cell population as in the case of the mesenchymal stem cell population of International Application WO 2018/067071. Thus, it would desirable to have at hand a quality control assay for the GMP production of the mesenchymal stem cell population of International Application WO 2018/067071.

In this context, it is known that mesenchymal stem cells, like any other biological material, have an intrinsic variability. For example, studies in cell biology of mesenchymal stem cells have identified a variety of factors that impact their longevity and expansion capacity. Issues such as source of donor tissue, donor age, environmental background, and isolation methods have been described to impact upon the overall quality of mesenchymal stem cells (see Paladino, et al. “Comparison between isolation protocols highlights intrinsic variability of human umbilical cord mesenchymal cells,” Cell and Tissue Banking, vol. 17, no. 1, pp. 123-136, (2016), https://doi.org/10.1007/s10561-015-9525-6). In addition, Paladino, et al. supra, 2016 addressed individual variability in mesenchymal stem cells by comparing three different isolation methods of MSC from umbilical cord for purposes of cell banking, and to identify if there were advantages in terms of cell viability, longevity in culture, expansion potential and differentiation capacities. The authors report that since the same samples were treated with at least two of the three studied protocols and under highly controlled experimental conditions, their results reveal that part of the observed variability is clearly intrinsic to each donor with readout in doubling time and longevity. In a further study, Paladino et al. (2017) “Intrinsic Variability Present in Wharton's Jelly Mesenchymal Stem Cells and T Cell Responses May Impact Cell Therapy”. Hindawi, Stem Cells International Volume 2017, Article ID 8492797, 12 pages, https://doi.org/10.1155/2017/8492797 showed that the gene expression of immunomodulatory molecules varied among samples of Wharton's Jelly mesenchymal stem cells (WJ-MSC) with no specific pattern present. In coculture, all WJ-MSCs were capable of inhibiting mitogen-activated CD3+ T cell proliferation, although to different degrees, and each PBMC responded with a different level of inhibition. The authors suggested that each WJ-MSC displays unique behavior, differing in patterns of cytokine mRNA expression and immunomodulatory capacity. The authors also assumed that variability between samples may play a role in the effectiveness of WJ-MSC employed therapeutically.

In light of these results, it is likely that MSC from the amniotic membrane of the umbilical cord also have an intrinsic variability with respect to the production of specific molecules which in turn may affect their suitability for therapeutic applications such as wound healing or diabetes. Thus, it would be of advantage to have at hand, a method for identifying a MSC population or MSC containing donor tissue that is suitable to be used for wound healing purposes, for example. Such a method would ideally be useful also for generating a master cell bank (which is necessary to produce a cellular therapy drug product) or for producing a stem cell population under cGMP conditions for subsequent pharmaceutical administration.

Accordingly, it is an object of the invention to provide a method that can, for instance be used, for identifying a donor for a MSC containing tissue or subsequently for a MSC population that is suitable to therapeutic applications such as wound healing.

### SUMMARY OF THE INVENTION

This object is accomplished by the methods and uses having the features of the independent claims.

In a first aspect, the invention provides a method of assessing the wound healing potency of a mesenchymal stem cell population, wherein the method comprises determining in a medium the level of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) secreted into the medium by the mesenchymal stem cell population.

In a second aspect, the invention provides a method of selecting a mesenchymal stem cell population for producing a stem cell population under cGMP conditions, wherein the method comprises determining in a medium the level of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) secreted into the a medium by the mesenchymal stem cell population.

In a third aspect, the invention provides a method of selecting a mesenchymal stem cell population for producing a stem cell population for subsequent pharmaceutical administration, wherein the method comprises determining in a medium the level of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) secreted into the medium by the mesenchymal stem cell population.

In a fourth aspect, the invention provides a method of selecting a mesenchymal stem cell population for generating a master cell bank, wherein the method comprises determining in a medium the level of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) secreted into the medium by the mesenchymal stem cell population.

In a fifth aspect, the invention provides a method of identifying a tissue suitable as starting material for producing a mesenchymal stem cell population for pharmaceutical use, wherein the method comprises determining the level of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) secreted into a medium by a sample of the tissue or a cell isolated from the tissue.

In a sixth aspect, the invention provides the use of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) protein for assessing the wound healing potency of a mesenchymal stem cell population.

In a seventh aspect, the invention provides the use of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) protein for selecting a mesenchymal stem cell population for producing a stem cell population under cGMP conditions.

In an eighth aspect, the invention provides the use of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) protein for selecting a mesenchymal stem cell population for producing a stem cell population for subsequent pharmaceutical administration.

In a ninth aspect, the invention provides the use of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) protein for selecting a mesenchymal stem cell population for generating a master cell bank.

In a tenth aspect, the invention provides the use of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) protein for identifying a tissue suitable as starting material for producing a mesenchymal stem cell population for pharmaceutical use.

In an eleventh aspect, the invention provides a method of identifying a medium suitable for inducing or improving wound healing properties of a mesenchymal stem cell population, wherein the method comprises determining in a medium the level of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) secreted into the medium by the mesenchymal stem cell population.

In a twelfth aspect, the invention provides the use of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) protein for identifying a medium suitable for inducing or improving wound healing properties of a mesenchymal stem cell population.

### BRIEF DESCRIPTION OF THE DRAWINGS

The invention will be better understood with reference to the detailed description when considered in conjunction with the non-limiting examples and the drawings, in which:

FIG. 1 shows a flow-diagram schematically representing the experimental steps of an illustrative example of a method of producing a therapeutic composition containing a MSC population with suitable wound healing potency. This method includes identifying a tissue suitable as starting material for producing a MSC population, assessing the wound healing potency of the MSC population, selecting a MSC population for producing a master cell bank and selecting a MSC population for pharmaceutical administration. The stem cells used in this example are isolated from the amniotic membrane of the umbilical cord—also referred herein to as cord lining stem cells (CLSC). This example starts with setting up a tissue bank comprising the umbilical cord tissue, which can be used as starting material for MSC cultivation. For this purpose, donor consent for the tissue donation may be obtained (stage 1). Further, maternal and newborn blood samples are screened for infectious diseases and the umbilical cords are tested for microbial contamination. Up to, for example, 100 umbilical cord samples may be collected in such a tissue bank. Tissue having found to be uncritical with respect to contamination or infectious diseases of the door is used for development cultures that are used to produce pure MSC lines (stage 2). For this purpose, outgrowths from about 10 individual amniotic membranes of the umbilical cord are cultivated for 0 to 2 passages for propagation. MSCs from Passage 2 (P2) are assessed by flow cytometry for MSC markers, and supernatants are assessed for cytokine production. The in-process release criteria for MSCs at stage 2 include >95% positive for CD73, CD90, CD105, and <5% positive for CD34, CD45 and HLA-DR, with >500 μg/ml production in vitro of Angiopoietin-1 and TGF-β, and >100 μg/ml of VEGF and HGF and negative sterility. Cell lines that meet the stage 2 in-process release criteria are then further propagated in a Terumo Quantum bioreactor for stage 3 of the manufacturing process. The in-process release criteria for MSCs at stage 3 include >95% positive for CD73, CD90, CD105, and <5% positive for CD34, CD45 and HLA-DR, tested for sterility, endotoxin, mycoplasma, human pathogen virus and adventitious virus. Mesenchymal cell lines (populations) that passed the tests are banked as master cell bank (stage 3). Master cell banks are thawed and seeded into cultures and release criteria are sterility, mycoplasma and endotoxin and finally 1×, 3× and 5×10⁶ MSCs that passed the tests are bottled in carrier medium such as 1 ml Hypothermosol® and stored at 2-8° C. before pharmaceutical administration (stage 4).

FIGS. 2A-2D show the result of a secretion level analysis of Ang-1, VEGF, HGF and TGF-β (here TGF-1) in 10 individual MSC populations obtained from 10 different umbilical cord donors cultivated as described for FIG. 1 post-stage 2. FIG. 2A shows the secretion level of Transforming Growth Factor beta (TGF-β) for the individual MSC populations 8049356, 8049358, 8049359, 8049364, 8049365, 8049369, 8049370, 8049372, 8049373 and 8049384. The secretion level of all 10 populations exceed the threshold value for TGF-β, which is about 500 pg/ml and indicated by the red line. FIG. 2B shows the secretion level of Hepatocyte Growth Factor (HGF) for the individual MSC populations 8049356, 8049358, 8049359, 8049364, 8049365, 8049369, 8049370, 8049372, 8049373 and 8049384. The secretion level of U.S. Pat. Nos. 8,049,365, 8,049,369, 8,049,372, 8,049,373 and 8,049,384 exceed the threshold value for HGF, which is about 100 pg/ml and indicated by the red line. FIG. 2C shows the secretion level of Angiopoietin 1 (Ang-1) for the individual MSC populations 8049356, 8049358, 8049359, 8049364, 8049365, 8049369, 8049370, 8049372, 8049373 and 8049384. The secretion level of all 10 populations exceed the threshold value for Ang-1, which is about 500 pg/ml and indicated by the red line.

FIG. 2D shows the secretion level of Vascular Endothelial Growth Factor (VEGF) for the individual MSC populations 8049356, 8049358, 8049359, 8049364, 8049365, 8049369, 8049370, 8049372, 8049373 and 8049384. Except for 8049358, 8049359 and 8049370, the secretion level of all populations exceed the threshold value for VEGF, which is about 100 pg/ml and indicated by the red line.

FIGS. 3A-3D show the assessment of cytokine secretion stability in a MSC population (8049372). For this purpose, the secretion level of TGF-β, HGF, Ang-1 and VEGF is each determined and compared in two separate samples of the same population post-stage 4. FIG. 3A shows the secretion level of TGF-β in two separate samples indicating a stable protein secretion of about 2230 μg/ml and 2419 μg/ml post-stage 4, respectively. FIG. 3B shows the secretion level of HGF in two separate samples indicating a stable protein secretion of about 933 μg/ml and 985 μg/ml post-stage 4, respectively. FIG. 3C shows the secretion level of Ang-1 in two separate samples indicating a stable protein secretion of about 1800 μg/ml and 1854 μg/ml post-stage 4, respectively. FIG. 3D shows the secretion level of VEGF in two separate samples indicating a stable protein secretion of about 210 μg/ml and 219 μg/ml post-stage 4, respectively.

FIGS. 4A-4D show the results of assays for identifying a medium suitable for inducing or improving wound healing properties of a mesenchymal stem cell population. MSCs obtained from the placenta, the Wharton's Jelly (WJ-MSC) and the amniotic membrane of the umbilical cord, also referred to as cord lining MSC (CL-MSC), were cultivated in different media suitable for cultivating MSCs (PTT4, PTT6 and DMEM/F12). The protein detection was performed in MSC supernatants and the analysis has been conducted using Luminex 200 and Xponent software. FIG. 4A summarizes the measurement of Ang-1. S1 denotes the highest standard used in the assay. Any samples that fall above are considered extrapolated (too concentrated). The graph depicts that all of CL-MSC, WJ-MSC and placental MSC produce much higher levels of Ang-1 when grown in PTT6 compared to when the MSC's were grown in PTT4 or DMEM/F12. FIG. 4B summarizes the measurement of VEGF in the analyzed supernatants of CL-MSC, WJ-MSC and placental MSC cultured in PTT6, PTT4 or DMEM/F12. S1 denotes the highest standard used in the assay. Any samples that fall above are considered extrapolated (too concentrated). As can be seen from the graph, all of CL-MSC, WJ-MSC and placental MSC produce much higher levels of VEGF when grown in PTT6 compared to when the MSC's were grown in PTT4 or DMEM/F12. FIG. 4C summarizes the measurement of HGF. The graph depicts that all of CL-MSC, WJ-MSC and placental MSC produce much higher levels of Ang-1 when grown in PTT6 compared to when the MSCs were grown in PTT4 or DMEM/F12.

FIG. 4D shows the singleplex measurement of TGF-β1. As can be seen from the graph, all of CL-MSC, WJ-MSC and placental MSC produce more TGF-β1 when grown in PTT6 than when grown in DMEM/F12.

### DETAILED DESCRIPTION OF THE INVENTION

The present invention is directed to several methods that are all suitable for validation and/or as quality control for various stages of a GMP manufacturing process of mesenchymal stem cell populations for therapeutic use. All these methods make use of determining in a medium the level of at least one protein selected from the group consisting of Ang-1, TGF-β, VEGF and HGF secreted into the medium by the mesenchymal stem cell population.

It has thus been surprisingly found here that determining the secretion level of Ang-1, TGF-β, VEGF and HGF in a medium of a MSC population is a suitable criterion for several aspects in a GMP manufacturing process of the MSC population. Determining the secretion level of Ang-1, TGF-β, VEGF and HGF in a medium in which an MSC population is cultivated or stored, may be used to assess the wound healing potency of the MSC population, to identify a (donor) tissue suitable as starting material for producing a MSC population with suitable wound healing potency, to select a MSC population for cGMP production, or to select a MSC population for subsequent pharmaceutical administration or to generate a master cell bank. Additionally, determining the secretion level of Ang-1, TGF-β, VEGF and HGF can be used for identifying a medium suitable for inducing or improving wound healing properties of a mesenchymal stem cell population.

It is noted here that involvement of Ang-1, TGF-β1, VEGF and HGF in the wound healing process is known to the person skilled in the art. For the involvement of Ang-1 (SEQ ID NO: 1) in wound healing, see, for example, Li et al. Stem Cell Research & Therapy 2013, 4:113 “Mesenchymal stem cells modified with angiopoietin-1 gene promote wound healing” or Bitto et al, “Angiopoietin-1 gene transfer improves the impaired wound healing of the genetically diabetic mice without increasing VEGF expression”, Clinical Science May 14, 2008, 114 (12) 707-718. In the study of Li et al, the Ang-1 gene was inserted into bone marrow mesenchymal stem cells and the results showed that that “Ang1-MSCs significantly promoted wound healing with increased epidermal and dermal regeneration, and enhanced angiogenesis compared with MSCs, Ad-Ang1 or sham treatment.” Notably, Li et al authors state that MSCs alone do not produce enough Ang-1 and for this reason, the authors inserted the Ang-1-gene into the MSC to come up with a genetically modified cell.

For the involvement of Transforming Growth Factor Beta, including TGF-1 (SEQ ID NO: 2, TGF-β2, and TGF-β3, in wound healing, in particular healing of chronic/non-healing wounds see for example, Ramirez et al. “The Role of TGFb Signaling in Wound Epithelialization” Advances In Wound Care, Volume 3, Number 7, 2013, 482-491 or Pakyari et al., Critical Role of Transforming Growth Factor Beta in Different Phases of Wound Healing, Advances In Wound Care, Volume 2, Number 5, 2012, 215-224.

For the involvement of VEGF (SEQ ID NO: 3) in wound healing, in particular healing of chronic/non-healing wounds, see for example Froget et al., Eur. Cytokine Netw., Vol. 14, March 2003, 60-64 or Bao et al., “The Role of Vascular Endothelial Growth Factor in Wound Healing” J Surg Res. 2009 May 15; 153 (2): 347-358.

Reverting to HGF (SEQ ID NO: 4) in wound healing, in particular healing of chronic/non-healing wounds, see for example, Yoshida et al., “Neutralization of Hepatocyte Growth Factor Leads to Retarded CutaneousWound Healing Associated with Decreased Neovascularization and Granulation Tissue Formation” J. Invest. Dermatol. 120:335-343, 2003, Li, Jin-Feng et al. “HGF Accelerates Wound Healing by Promoting the Dedifferentiation of Epidermal Cells through β 1-Integrin/ILK Pathway.” BioMed Research International 2013 (2013): 470418 or Conway et al, “Hepatocyte growth factor regulation: An integral part of why wounds become chronic”. Wound Rep Reg (2007) 15 683-692.

Wound healing potency may describe the capability, ability, competence or effectiveness to facilitate or accelerate wound healing. In the present invention, a MSC population is considered to have sufficient wound healing potency, for example, or a tissue is considered to be suitable for be suitable as starting material for producing a pharmaceutically suitable MSC population, if the secretion level (also referred to as concentration) of one, two, three or all four of Ang-1, TGF-β, VEGF and HGF is equal or higher than a threshold specific for each of these protein as defined herein (cf. also Example 2). Thus, assessing wound healing comprises determining the secretion level of Ang-1, TGF-β, VEGF and HGF and subsequent determination of whether the specific thresholds are reached or exceeded. Assessing the wound healing potency of a MSC population may be performed during different stages of MSC cultivation. The wound healing potency may be assessed on tissue directly before MSC cultivation to identify a tissue suitable as starting material for producing a MSC population having wound healing potency and thus may being suitable for subsequent pharmaceutical use. To be suitable for pharmaceutical application, a MSC population may have to be produced under current Good Manufacturing Practice (cGMP) conditions. Thus, determining the secretion level of Ang-1, TGF-β, VEGF and HGF before producing a MSC population may be suitable to select a MSC population for producing a MSC population under cGMP conditions. Further, a MSC population can be selected for subsequent pharmaceutical administration using the methods described herein.

A MSC population selected according to the present invention may also be used for generating a master cell bank. In this context, the selected MSC population may be further characterized and tested for integrity and contaminants such as bacteria, fungi, mycoplasmas and viruses before cryopreservation. Once set up, a MSC master cell bank may allow an expansion of a specific MSC population to form cultures for further research or production processes whenever needed. For example, a MSC master cell bank comprising MSC with wound healing properties may allow the expansion of a specific MSC population may secreting an amount of Ang-1, TGF-β, VEGF and HGF that is ideal for wound healing.

The secretion level of Ang-1, TGF-β, VEGF and/r HGF is used as selection criterion in the methods described herein. A secretion level equaling or exceeding a threshold may, for example, indicate (i) potency for wound healing of a MSC population or (ii) a tissue or an isolated cell population being suitable as starting material for producing a MSC population. In the present invention, the threshold for Ang-1 may be about 100 pg/ml, about 200 pg/ml, about 300 pg/ml, about 400 pg/ml, about 500 pg/ml, about 600 pg/ml, about 700 pg/ml, about 800 pg/ml, about 900 pg/ml or about 1000 pg/ml. Preferably, for Ang-1 the threshold is about 500 pg/ml. The threshold for TGF-β may be about 100 pg/ml, about 200 pg/ml, about 300 pg/ml, about 400 pg/ml, about 500 pg/ml, about 600 pg/ml, about 700 pg/ml, about 800 pg/ml, about 900 pg/ml or about 1000 pg/ml. Preferably, for TGF-β the threshold is about 500 pg/ml. Regarding VEGF, the threshold may be about 80 pg/ml, about 100 pg/ml, about 120 pg/ml, about 140 pg/ml, about 160 pg/ml, about 180 pg/ml or about 200 pg/ml, wherein the threshold for VEGF preferably is about 100 pg/ml. Turning to HGF the threshold may be about 80 pg/ml, about 100 pg/ml, about 120 pg/ml, about 140 pg/ml, about 160 pg/ml, about 180 pg/ml or about 200 pg/ml. Preferably, for HGF the threshold is about 100 pg/ml.

In one example of the present invention, the following threshold levels (values) are used:
a threshold of about 400 pg/ml for Angiopoietin 1 (Ang-1)
a threshold of about 400 pg/ml for Transforming Growth Factor beta (TGF-β)
a threshold of about 80 pg/ml for Vascular Endothelial Growth Factor (VEGF)
a threshold of about 80 pg/ml for Hepatocyte Growth Factor (HGF).

In another example of the present invention, the following threshold levels (values) are used:
a threshold of about 500 pg/ml for Angiopoietin 1 (Ang-1)
a threshold of about 500 pg/ml for Transforming Growth Factor beta (TGF-β)
a threshold of about 100 pg/ml for Vascular Endothelial Growth Factor (VEGF)
a threshold of about 100 pg/ml for Hepatocyte Growth Factor (HGF).

In these two examples, the secretion level of all four proteins equal or exceed their respective threshold value of the secretion level/concentration (cf. Example 2) in order to, for example, consider a MSC population to have suitable wound healing properties or to consider a tissue to be a suitable starting material for producing a pharmaceutically suitable MSC population. It is noted here that the concentrations determined in the present invention and thus the threshold levels are preferably absolute concentrations.

Any pharmaceutically suitable MSC population can be used in the present invention. Thus, the MSC population may be derived from any mammalian tissue or compartment/body part known to contain MSC. In illustrative examples, the MSC population may be a MSC population of the umbilical cord, a placental MSC population, a MSC population of the cord-placenta junction, a MSC population of the cord blood, a MSC of the bone marrow, or an adipose-tissue derived MSC population. The MSC population of the umbilical cord may be (derived) from any compartment of umbilical cord tissue that contains MSCs such as the amnion, a perivascular MSC population, a MSC population of Wharton's jelly, a MSC population of the amniotic membrane of umbilical cord but also a mixed MSC population of the umbilical cord, meaning a population of MSCs that includes stem cells of two or more of these compartments. MSCs of these compartments and their isolation therefrom are known to the person skilled in the art and are described, for example, by Subramanian et al “Comparative Characterization of Cells from the Various Compartments of the Human Umbilical Cord Shows that the Wharton's Jelly Compartment Provides the Best Source of Clinically Utilizable Mesenchymal Stem Cells”, PLOS ONE 10 (6): e0127992, 2015 and the references cited therein, Van Pham et al. “Isolation and proliferation of umbilical cord tissue derived mesenchymal stem cells for clinical applications”, Cell Tissue Bank (2016) 17:289-302, 2016. A mixed MSC population of the umbilical cord can, for example, be obtained by removing the arteries and veins from the umbilical cord tissue, cutting the remaining tissue and the Wharton's jelly into piece and cultivating the umbilical cord tissue (by tissue explant) in the culture medium of the present invention. A mixed MSC population of the umbilical cord may also be obtained by culturing entire umbilical cord tissue with intact umbilical vessels as tissue explant under the conditions (cultivation in serum-supplemented DMEM with 10% fetal bovine serum, 10% horse serum, and 1% Penicillin/Streptomycin) as described by Schugar et al. “High harvest yield, high expansion, and phenotype stability of CD146 mesenchymal stromal cells from whole primitive human umbilical cord tissue. Journal of biomedicine & biotechnology. 2009; 2009:789526”. In this context, it is noted that a MSC population of the cord-placenta junction can be isolated as described by Beeravolu et al. “Isolation and Characterization of Mesenchymal Stromal Cells from Human Umbilical Cord and Fetal Placenta.” J Vis Exp. 2017; (122): 55224. In examples of the present invention, the MSC population if derived from an umbilical cord or the amniotic membrane of the umbilical cord (cf. Example 1-4). A MSC population of the amniotic membrane of umbilical cord may be highly defined and homogenous. Thus, in one embodiment of the present invention, a mesenchymal stem cell population as described in International Application WO 2018/067071 is used. Thus, in typical examples of the method at least about 90% or more, about 91% or more, about 92% or more, about 93% or more, about 94% or more, about 95% or more, about 96% or more, about 97% or more, about 98% or more about 99% or more of the MSCs express the following markers: CD73 (SEQ ID NO. 5), CD90 (SEQ ID NO. 6) and CD105 (SEQ ID NO. 7). In addition, in these examples at least about 90% or more, about 91% or more, about 92% or more, about 93% or more, about 94% or more, about 95% or more, about 96% or more, about 97% or more, about 98% or more, about 99% or more of the MSCs may lack expression of the following markers: CD34 (SEQ ID NO. 8), CD45 (SEQ ID NO. 9) and HLA-DR (SEQ ID NO. 10). In particular examples, about 97% or more, about 98% or more, or about 99% or more of the MSC population express CD73, CD90 and CD105 while lacking expression of CD34, CD45 and HLA-DR. In preferred examples at least about 91% or more, about 92% or more, about 93% or more, about 94% or more, about 95% or more, about 96% or more, about 97% or more, about 98% or more about 99% or more cells of the MSC population express each of CD73, CD90 and CD105 while at least about 90% or more, about 91% or more, about 92% or more, about 93% or more, about 94% or more, about 95% or more, about 96% or more, about 97% or more, about 98% or more about 99% or more of the MSC may lack expression of CD34, CD45 and HLA-DR. In particular examples about 97% or more, about 98% or more, or about 99% or more of the MSC population express CD73, CD90 and CD105 while lacking expressing of CD34, CD45 and HLA-DR.

In the present invention, the level of Ang-1, TGF-β, VEGF and HGF is typically determined in the supernatant of the medium in which a MSC population is either stored, transported or cultivated in. An MSC population may be stored for a long or for a short term. Examples for long term storage media include but are not limited to glycerol and trehalose, allowing a storage at about −80° C., or a cryoprotectant such as dimethylsulfoxide (DMSO), allowing a storage at about −195° C. Short term storage may comprise transport to an administration site (such as an doctor office's or hospital) and/or storage for a period of time till the MSC population will be administered to a subject. The excipient HypoThermosol® is an illustrative example for a short term storage medium. This preservation medium is suitable for transportation, allowing a MSC storage at about 2 to 8° C. Another example for a medium suitable for transportation is Plasmalyte. Examples for media suitable for MSC cultivation may comprise, but are not limited to commercial available medium such as CTS StemPro MSC SFM, MesenPRO RS Medium, StemPro MSC SFM XenoFree. In one example of the present invention, the MSC cell culture medium may be the cultivation medium PTT6 that is described in International Application WO 2018/067071. In a with the disclosure of International Application WO 2018/067071 the MSC cell culture medium may thus comprise Dulbecco's modified eagle medium (DMEM), Ham's F12 Medium (F12), a serum free basal medium such as M171 and Fetal Bovine Serum (FBS). Thus, in one example, the medium may comprise DMEM in a final concentration of about 55 to 65% (v/v), F12 in a final concentration of about 5 to 15% (v/v), M171 in a final concentration of about 15 to 30% (v/v) and FBS in a final concentration of about 1 to 8% (v/v). The value of “% (v/v)” as used herein refers to the volume of the individual component relative to the final volume of the medium. This means, if DMEM is, for example, present in the medium a final concentration of about 55 to 65% (v/v), 1 liter of medium contains about 550 to 650 ml DMEM. In other examples, the medium may comprise DMEM in a final concentration of about 57.5 to 62.5% (v/v), F12 in a final concentration of about 7.5 to 12.5% (v/v), M171 in a final concentration of about 17.5 to 25.0% (v/v) and FBS in a final concentration of about 1.75 to 3.5% (v/v). In further examples, the medium may comprise DMEM in a final concentration of about 61.8% (v/v), F12 in a final concentration of about 11.8% (v/v), M171 in a final concentration of about 23.6% (v/v) and FBS in a final concentration of about 2.5% (v/v). In addition to the above-mentioned components, the medium may comprise supplements that are advantageous for MSC cultivation. In the present invention, the MSC culture medium may, for example, comprise Epidermal Growth Factor (EGF). If present, EGF may be present in the culture medium in a final concentration of about 1 ng/ml to about 20 ng/ml. In some of these examples, the culture medium may comprise EGF in a final concentration of about 10 ng/ml. The culture medium of the present invention may also comprise insulin. If present, insulin may be present in a final concentration of about 1 μg/ml to 10 μg/ml. In some of these examples, the culture medium may comprise Insulin in a final concentration of about 5 μg/ml. The culture medium may further comprise at least one of the following supplements: adenine, hydrocortisone, and 3,3′,5-Triiodo-L-thyronine sodium salt (T3). In such examples, the culture medium may comprise all three of adenine, hydrocortisone, and 3,3′,5-Triiodo-L-thyronine sodium salt (T3). In these examples, the culture medium may comprise adenine in a final concentration of about 0.05 to about 0.1 μg/ml adenine, hydrocortisone in a final concentration of about 1 to about 10 μg/ml hydrocortisone and/or 3,3′,5-Triiodo-L-thyronine sodium salt (T3) in a final concentration of about 0.5 to about 5 ng/ml. In this context, it is noted that by cultivating a MSC population in a medium as described herein, the expression and/or secretion of at least one protein selected from the group consisting of Ang-1, TGF-β, VEGF and HGF may be increased.

In the present method, it may be required to subject the medium to centrifugation for determining the concentration of Ang-1, TGF-β, VEGF and HGF in the medium that contains the MSC, typically after a suitable period of time. In this context, a suitable period of time may be any incubation period (if the cell population is, for example stored, or transported in a storage or transport medium such as Hypothermosol) or cultivation period (if the cell population is cultivated in culture medium) are suitable for a MSC population to secrete proteins in a detectable amount. In an illustrative example, a suitable period of time may be an incubation or cultivation period of about 6 h, about 12 h, about 18 h, about 24 h, about 30 h, about 36 h, about 42 h, about 46 h, about 48 h, about 50 h or about 54 h. After centrifugation, the supernatant of the centrifuged medium may be subjected to an immunoassay to determine the levels/concentration of the secreted proteins. Any immunoassay suitable to detect one or multiple secreted proteins in a medium can be applied in the present invention. Illustrative examples of suitable immunoassays for detecting protein in a medium are an Enzyme-linked Immunosorbent Assay (ELISA) or a singleplex assay. Singleplex assays (that are commercially available, for example, from BioVendor, Brno, Czech Republic under the trade name Q-Plex or from R&D Systems Inc, Minneapolis, USA) may be carried out by placing two spots consisting of capture antibodies in a defined array to the bottom of each well of a 96-well ELISA plate (in addition to the assay spot, the second spot is a positive control spot for assuring proper assay procedure). An example of a suitable assay that detects multiple proteins in a medium is a multiplex assay. In such a multiplex assay multiple analytes can be immobilized on a solid surface such as an ELISA plate, which separates the analytes spatially. Alternatively, a multiplex assay may also be carried out using analytes that have been immobilized on beads or particles. In such a case, the assays for each analyte employs different bead/particles. In illustrative examples, a bead based multiplex assay may be used to detect secreted Ang-1, TGF-β, VEGF and HGF in a medium (cf. Example 1 and Example 4). Such multiplex assay systems for simultaneously detecting and quantifying multiple target analytes in complex samples such as cell culture medium are commercially available, for example, from R&D Systems Inc, Minneapolis, USA as Luminex® Assays and Luminex® High Performance Assays.

The present invention is also directed to a use of at least one protein selected from the group consisting of Ang-1, TGF-β, VEGF and HGF for assessing the wound healing potency of a MSC population. Further, the present invention is directed to a use of at least one protein selected from the group consisting of Ang-1, TGF-β, VEGF and HGF for selecting a MSC population for producing a stem cell population under cGMP conditions. Consequently, the present invention is directed to a use of at least one protein selected from the group consisting of Ang-1, TGF-β, VEGF and HGF for selecting a MSC population for producing a stem cell population for subsequent pharmaceutical administration. The present invention is further directed to a use of at least one protein selected from the group consisting of Ang-1, TGF-β, VEGF and HGF for selecting a MSC population for generating a master cell bank.

Additionally, the present invention is directed to a use of at least one protein selected from the group consisting of Ang-1, TGF-β, VEGF and HGF for selecting a MSC population for identifying a tissue suitable as starting material for producing a MSC population for pharmaceutical use. In the present invention, such tissue may be any mammalian tissue or compartment/body part known to contain MSCs. Examples for such tissue include, but are not limited to bone marrow, adipose-tissue, placental tissue, tissue of the cord-placenta junction, umbilical cord tissue such as Wharton's Jelly, the amniotic membrane of the umbilical cord to name only a few known tissue sources for MSCs. In an example, the tissue is an umbilical cord or the amniotic membrane of the umbilical cord and the MSC population produced therefrom may be a MSC population of the amniotic membrane of umbilical cord.

For determining whether a tissue, for example, from a particular donor is suitable as a starting material for producing an MSC population for pharmaceutical use, the tissue can, for example, be directly cultivated as tissue explant. For such tissue explant, a sample of the respective tissue (for example, Wharton's Jelly, the placental amnion or the amniotic membrane of the umbilical cord) can be placed in tissue culture dishes and cultivated in a suitable cultivation/growth medium as described here (cf. also U.S. Pat. No. 9,085,755, or 9,737,568 in this respect). Cell outgrowth from the tissue (migration of MSCs out of the tissue onto the surface of the culture dish) will then occur after an suitable period of cultivation and the culture medium can then be analyzed for the secretion of Ang-1, TGF-β, VEGF and HGF. Alternatively, the MSC population can first be isolated from the chosen tissue using a known isolation method and the isolated MSC population can then be cultivated in a suitable medium and checked for the secretion of Ang-1, TGF-β, VEGF and HGF. Independently from the tissue, the use of Ang-1, TGF-β, VEGF and HGF for the methods described herein may comprise determining in the medium at least one, at least two, at least three or all four of said proteins secreted into the medium by the MSC population or by a tissue sample or by a cell isolated from the tissue sample.

The present invention is further directed to a method of identifying a medium suitable for suitable for inducing or improving wound healing properties of a mesenchymal stem cell population. Also this method comprises determining the level of at least one protein selected from the group consisting of Ang-1, TGF-β, VEGF and HGF secreted into the cell culture medium by the MSC population. Consequently, the present invention is also directed to the use of at least one protein for identifying a medium suitable suitable for inducing or improving wound healing properties of a mesenchymal stem cell. This use may comprise determining in the cell culture medium the level of at least two, at least three or all four proteins selected from the group consisting of Ang-1, TGF-β, VEGF and HGF secreted into the cell culture medium by the mesenchymal stem cell population.

The invention will be further illustrated by the following non-limiting Experimental Examples.

Sequences of polypeptides disclosed herein are depicted in Table 1.

TABLE 1

| SEQ | | |
|---|---|---|
| ID | | |
| NO. | Polypeptide | Sequence |
| 1 | Human Ang-1 | MTVFLSFAFLAAILTHIGCSNQRRSPENSGRRYNRIQHGQCAYTFILPEHD |
| | Uniprot no: | GNCRESTTDQYNTNALQRDAPHVEPDFSSQKLQHLEHVMENYTQWLQ |
| | Q15389 | KLENYIVENMKSEMAQIQQNAVQNHTATMLEIGTSLLSQTAEQTRKLTD |
| | version | VETQVLNQTSRLEIQLLENSLSTYKLEKQLLQQTNEILKIHEKNSLLEHKI |
| | number 2 | LEMEGKHKEELDTLKEEKENLQGLVTRQTYIIQELEKQLNRATTNNSVL |
| | as of | QKQQLELMDTVHNLVNLCTKEGVLLKGGKREEEKPFRDCADVYQAGF |
| | Jan. 1, 1998 | NKSGIYTIYINNMPEPKKVFCNMDVNGGGWTVIQHREDGSLDFQRGWK |
| | | EYKMGFGNPSGEYWLGNEFIFAITSQRQYMLRIELMDWEGNRAYSQYD |
| | | RFHIGNEKQNYRLYLKGHTGTAGKQSSLILHGADFSTKDADNDNCMCK |
| | | CALMLTGGWWFDACGPSNLNGMFYTAGQNHGKLNGIKWHYFKGPSYS |
| | | LRSTTMMIRPLDF |
| 2 | Human | MEAAVAAPRPRLLLLVLAAAAAAAAALLPGATALQCFCHLCTKDNFTC |
| | TGFbeta1 | VTDGLCFVSVTETTDKVIHNSMCIAEIDLIPRDRPFVCAPSSKTGSVTTTY |
| | Uniprot no: | CCNQDHCNKIELPTTVKSSPGLGPVELAAVIAGPVCFVCISLMLMVYICH |
| | P36897 | NRTVIHHRVPNEEDPSLDRPFISEGTTLKDLIYDMTTSGSGSGLPLLVQRT |
| | version | IARTIVLQESIGKGRFGEVWRGKWRGEEVAVKIFSSREERSWFREAEIYQ |
| | number 1 | TVMLRHENILGFIAADNKDNGTWTQLWLVSDYHEHGSLFDYLNRYTVT |
| | as of | VEGMIKLALSTASGLAHLHMEIVGTQGKPAIAHRDLKSKNILVKKNGTC |
| | Jun. 1, 1994 | CIADLGLAVRHDSATDTIDIAPNHRVGTKRYMAPEVLDDSINMKHFESF |
| | | KRADIYAMGLVFWEIARRCSIGGIHEDYQLPYYDLVPSDPSVEEMRKVV |
| | | CEQKLRPNIPNRWQSCEALRVMAKIMRECWYANGAARLTALRIKKTLS |
| | | QLSQQEGIKM |
| 3 | Human | MNFLLSWVHWSLALLLYLHHAKWSQAAPMAEGGGQNHHEVVKFMDV |
| | VEGFA | YQRSYCHPIETLVDIFQEYPDEIEYIFKPSCVPLMRCGGCCNDEGLECVPT |
| | Uniprot no: | EESNITMQIMRIKPHQGQHIGEMSFLQHNKCECRPKKDRARQEKKSVRG |
| | P15692 | KGKGQKRKRKKSRYKSWSVYVGARCCLMPWSLPGPHPCGPCSERRKH |
| | version | LFVQDPQTCKCSCKNTDSRCKARQLELNERTCRCDKPRR |
| | number 2 | |
| | as of Nov. | |
| | 16, 2001 | |
| 4 | Human HGF | MWVTKLLPALLLQHVLLHLLLLPIAIPYAEGQRKRRNTIHEFKKSAKTTL |
| | Uniprot no: | IKIDPALKIKTKKVNTADQCANRCTRNKGLPFTCKAFVFDKARKQCLWF |
| | P14210 | PFNSMSSGVKKEFGHEFDLYENKDYIRNCIIGKGRSYKGTVSITKSGIKCQ |
| | version | PWSSMIPHEHSFLPSSYRGKDLQENYCRNPRGEEGGPWCFTSNPEVRYE |
| | number 2 | VCDIPQCSEVECMTCNGESYRGLMDHTESGKICQRWDHQTPHRHKFLPE |
| | as of | RYPDKGFDDNYCRNPDGQPRPWCYTLDPHTRWEYCAIKTCADNTMND |
| | Aug. 1, 1991 | TDVPLETTECIQGQGEGYRGTVNTIWNGIPCQRWDSQYPHEHDMTPENF |
| | | KCKDLRENYCRNPDGSESPWCFTTDPNIRVGYCSQIPNCDMSHGQDCYR |
| | | GNGKNYMGNLSQTRSGLTCSMWDKNMEDLHRHIFWEPDASKLNENYC |
| | | RNPDDDAHGPWCYTGNPLIPWDYCPISRCEGDTTPTIVNLDHPVISCAKT |
| | | KQLRVVNGIPTRTNIGWMVSLRYRNKHICGGSLIKESWVLTARQCFPSR |
| | | DLKDYEAWLGIHDVHGRGDEKCKQVLNVSQLVYGPEGSDLVLMKLAR |
| | | PAVLDDFVSTIDLPNYGCTIPEKTSCSVYGWGYTGLINYDGLLRVAHLYI |
| | | MGNEKCSQHHRGKVTLNESEICAGAEKIGSGPCEGDYGGPLVCEQHKM |
| | | RMVLGVIVPGRGCAIPNRPGIFVRVAYYAKWIHKIILTYKVPQS |
| 5 | CD73 | MCPRAARAPATLLLALGAVLWPAAGAWELTILHTNDVHSRLEQTSEDS |
| | identifier | SKCVNASRCMGGVARLFTKVQQIRRAEPNVLLLDAGDQYQGTIWFTVY |
| | P21589 of | KGAEVAHFMNALRYDAMALGNHEFDNGVEGLIEPLLKEAKFPILSANIK |
| | Uniprot, | AKGPLASQISGLYLPYKVLPVGDEVVGIVGYTSKETPFLSNPGTNLVFED |
| | version | EITALQPEVDKLKTLNVNKIIALGHSGFEMDKLIAQKVRGVDVVVGGHS |
| | number 1 as of | NTFLYTGNPPSKEVPAGKYPFIVTSDDGRKVPVVQAYAFGKYLGYLKIEFD |
| | May 1, 1991: | ERGNVISSHGNPILLNSSIPEDPSIKADINKWRIKLDNYSTQELGKTIVY |
| | | LDGSSQSCRFRECNMGNLICDAMINNNLRHTDEMFWNHVSMCILNGGG |
| | | IRSPIDERNNGTITWENLAAVLPFGGTFDLVQLKGSTLKKAFEHSVHRYG |
| | | QSTGEFLQVGGIHVVYDLSRKPGDRVVKLDVLCTKCRVPSYDPLKMDE |
| | | VYKVILPNFLANGGDGFQMIKDELLRHDSGDQDINVVSTYISKMKVIYP |
| | | AVEGRIKFSTGSHCHGSFSLIFLSLWAVIFVLYQ |
| 6 | CD90 | MNLAISIALLLTVLQVSRGQKVTSLTACLVDQSLRLDCRHENTSSSPIQY |
| | identifier | EFSLTRETKKHVLFGTVGVPEHTYRSRTNFTSKYNMKVLYLSAFTSKDE |
| | P04216 of | GTYTCALHHSGHSPPISSQNVTVLRDKLVKCEGISLLAQNTSWLLLLLLS |
| | Uniprot, | LSLLQATDFMSL |
| | version | |
| | number 2 as of | |
| | May 2, 2002: | |
| 7 | CD105 | MDRGTLPLAVALLLASCSLSPTSLAETVHCDLQPVGPERGEVTYTTSQVS |
| | identifier | KGCVAQAPNAILEVHVLFLEFPTGPSQLELTLQASKQNGTWPREVLLVL |
| | P17813 of | SVNSSVFLHLQALGIPLHLAYNSSLVTFQEPPGVNTTELPSFPKTQILEWA |
| | Uniprot, | AERGPITSAAELNDPQSILLRLGQAQGSLSFCMLEASQDMGRTLEWRPRT |
| | version | PALVRGCHLEGVAGHKEAHILRVLPGHSAGPRTVTVKVELSCAPGDLDA |
| | number 2 as of | VLILQGPPYVSWLIDANHNMQIWTTGEYSFKIFPEKNIRGFKLPDTPQGL |
| | Jul. 15, 1998: | LGEARMLNASIVASFVELPLASIVSLHASSCGGRLQTSPAPIQTTPPKDTC |
| | | SPELLMSLIQTKCADDAMTLVLKKELVAHLKCTITGLTFWDPSCEAEDR |
| | | GDKFVLRSAYSSCGMQVSASMISNEAVVNILSSSSPQRKKVHCLNMDSL |
| | | SFQLGLYLSPHFLQASNTIEPGQQSFVQVRVSPSVSEFLLQLDSCHLDLGP |
| | | EGGTVELIQGRAAKGNCVSLLSPSPEGDPRFSFLLHFYTVPIPKTGTLSCT |
| | | VALRPKTGSQDQEVHRTVFMRLNIISPDLSGCTSKGLVLPAVLGITFGAF |
| | | LIGALLTAALWYIYSHTRSPSKREPVVAVAAPASSESSSTNHSIGSTQSTP |
| | | CSTSSMA |
| 8 | CD34 | MLVRRGARAGPRMPRGWTALCLLSLLPSGFMSLDNNGTATPELPTQGTFS |
| | identifier | NVSTNVSYQETTTPSTLGSTSLHPVSQHGNEATTNITETTVKFTSTSVIT |
| | P28906 of | SVYGNTNSSVQSQTSVISTVFTTPANVSTPETTLKPSLSPGNVSDLSTTS |
| | Uniprot, | TSLATSPTKPYTSSSPILSDIKAEIKCSGIREVKLTQGICLEQNKTSSCA |
| | version number | EFKKDRGEGLARVLCGEEQADADAGAQVCSLLLAQSEVRPQCLLLVLANR |
| | 2 as of | TEISSKLQLMKKHQSDLKKLGILDFTEQDVASHQSYSQKTLIALVTSGAL |
| | Jul. 15, 1998: | LAVLGITGYFLMNRRSWSPTGERLGEDPYYTENGGGQGYSSGPGTSPEAQ |
| | | GKASVNRGAQENGTGQATSRNGHSARQHVVADTEL |
| 9 | CD45 | MYLWLKLLAFGFAFLDTEVFVTGQSPTPSPTGLTTAKMPSVPLSSDPLPT |
| | identifier | HTTAFSPASTFERENDFSETTTSLSPDNTSTQVSPDSLDNASAFNTTGVSS |
| | P08575 of | VQTPHLPTHADSQTPSAGTDTQTFSGSAANAKLNPTPGSNAISDVPGERS |
| | Uniprot, | TASTFPTDPVSPLTTTLSLAHHSSAALPARTSNTTITANTSDAYLNASETT |
| | version number | TLSPSGSAVISTTTIATTPSKPTCDEKYANITVDYLYNKETKLFTAKLNVN |
| | 2 as of | ENVECGNNTCTNNEVHNLTECKNASVSISHNSCTAPDKTLILDVPPGVEK |
| | Jul. 19, 2003: | FQLHDCTQVEKADTTICLKWKNIETFTCDTQNITYRFQCGNMIFDNKEIK |
| | | LENLEPEHEYKCDSEILYNNHKFTNASKIIKTDFGSPGEPQIIFCRSEAAH |
| | | QGVITWNPPQRSFHNFTLCYIKETEKDCLNLDKNLIKYDLQNLKPYTKYV |
| | | LSLHAYIIAKVQRNGSAAMCHFTTKSAPPSQVWNMTVSMTSDNSMHVK |
| | | CRPPRDRNGPHERYHLEVEAGNTLVRNESHKNCDFRVKDLQYSTDYTFK |
| | | AYFHNGDYPGEPFILHHSTSYNSKALIAFLAFLIIVTSIALLVVLYKIYDL |
| | | HKKRSCNLDEQQELVERDDEKQLMNVEPIHADILLETYKRKIADEGRLF |
| | | LAEFQSIPRVFSKFPIKEARKPFNQNKNRYVDILPYDYNRVELSEINGDAG |
| | | SNYINASYIDGFKEPRKYIAAQGPRDETVDDFWRMIWEQKATVIVMVTR |
| | | CEEGNRNKCAEYWPSMEEGTRAFGDVVVKINQHKRCPDYIIQKLNIVNK |
| | | KEKATGREVTHIQFTSWPDHGVPEDPHLLLKLRRRVNAFSNFFSGPIVVH |
| | | CSAGVGRTGTYIGIDAMLEGLEAENKVDVYGYVVKLRRQRCLMVQVE |
| | | AQYILIHQALVEYNQFGETEVNLSELHPYLHNMKKRDPPSEPSPLEAEFQ |
| | | RLPSYRSWRTQHIGNQEENKSKNRNSNVIPYDYNRVPLKHELEMSKESE |
| | | HDSDESSDDDSDSEEPSKYINASFIMSYWKPEVMIAAQGPLKETIGDFWQ |
| | | MIFQRKVKVIVMLTELKHGDQEICAQYWGEGKQTYGDIEVDLKDTDKS |
| | | STYTLRVFELRHSKRKDSRTVYQYQYTNWSVEQLPAEPKELISMIQVVK |
| | | QKLPQKNSSEGNKHHKSTPLLIHCRDGSQQTGIFCALLNLLESAETEEVV |
| | | DIFQVVKALRKARPGMVSTFEQYQFLYDVIASTYPAQNGQVKKNNHQE |
| | | DKIEFDNEVDKVKQDANCVNPLGAPEKLPEAKEQAEGSEPTSGTEGPEH |
| | | SVNGPASPALNQGS |
| 10 | HLA-DR | MAISGVPVLGFFIIAVLMSAQESWAIKEEHVIIQAEFYLNPDQSGEFMFDF |
| | identifier | DGDEIFHVDMAKKETVWRLEEFGRFASFEAQGALANIAVDKANLEIMT |
| | P01903 of | KRSNYTPITNVPPEVTVLTNSPVELREPNVLICFIDKFTPPVVNVTWLRNG |
| | Uniprot, | KPVTTGVSETVFLPREDHLFRKFHYLPFLPSTEDVYDCRVEHWGLDEPLL |
| | version number | KHWEFDAPSPLPETTENVVCALGLTVGLVGIIIGTIFIIKGVRKSNAAERR |
| | 1 as of | GPL |
| | Jul. 21, 1986: | |

### EXPERIMENTAL EXAMPLES

### Example 1: Identifying a Suitable Umbilical Cord Containing MSC, Assessing and Selecting a MSC Population Obtained from the Umbilical Cord for Generating a Pharmaceutical Composition Suitable for Wound Healing

MSCs were derived from fresh umbilical cord tissue collected at the University of Colorado Hospital.

### Stage 1: Tissue Bank

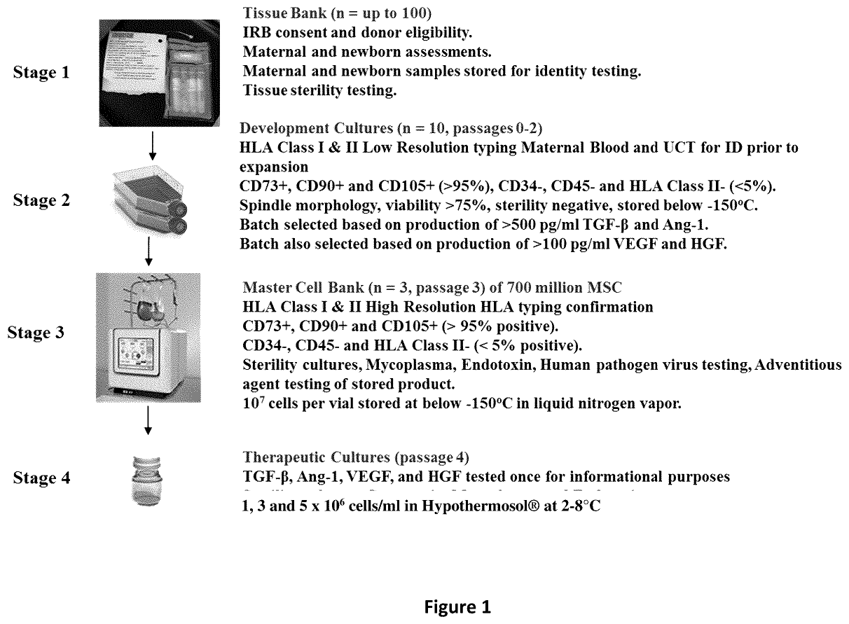

In a first step, umbilical cords are collected, usually after the Institutional Review Board (IRB) consent is obtained from the tissue donor. Fresh collected umbilical cord is cut into 1 to 2 mm³ sections, controlled-rate frozen in vials containing 4 to 5 segments and stored in quarantine in liquid nitrogen (LN2) vapor phase at −196° C. (cf. FIG. 1). Maternal blood samples, collected within seven days of delivery, are screened for infectious diseases and the tissues are tested for microbial contamination. The in-process release criteria for Stage 1 include infectious disease negative for all tests except CMV, sterility negative, and maternal questionnaire acceptable. Although the umbilical cords are naturally contaminated by vaginal flora during delivery, the collection in antibiotics will render some of them sterile. Up to 100 umbilical cord samples can be collected in such a tissue bank. The sterile tissues will be used in the Stage 2.

### Stage 2: Development Cultures

Developmental cultures are the outgrowths from the umbilical tissues that are used to produce pure MSC lines. 10 tissue segments are placed individually in 6-well plates and propagated for approximately 10-20 days to generate Passage 0 (P0) cells. P0 cells are seeded in 175 cm² flask and cultivated to obtain Passage 1 (P1) cells. P1 cells are frozen at 1-3×10⁶ cells/vial in CryoStor 5 or seeded for culture. Passage 1 cells are seeded at 2-3×10⁵ cells/175 cm² flask. During propagation, the cell morphology is recorded. Cells from Passage 2 (P2) are assessed by flow cytometry for MSC markers, and supernatants are assessed for cytokine production. In more detail, P2 cells are subjected to a multiplex analysis (R&D Systems/Bio-techne cat. #LXSAHM) with the following analytes Ang-1, VEGF, HGF and a singleplex assay was carried out with TGF-β. The assays have been carried out as follows:

### Multiplex Assay:

(i) A standard was prepared by combining 100 μl of each standard into a single microcentrifuge tube which already contained the appropriate volume of complete PTT6 medium to make up the total volume to 1000 μl. Standard S1 contained all the multiplex standards combined together in a single vial. To make a 3-fold serial dilution of S1, 200 μl of complete PTT6 medium were pipetted into each of five 1.5 ml polypropylene tubes labeled S2-S6. Then, 100 μl were transferred from S1 to S2. After vortexing, 100 μl were transferred from S2 to S3. The procedure was continued up to S6. PTT6 complete medium served as the Blank.
(ii) Beads were prepared by gently vortexing the vial to re-suspend. It was important to be careful not to invert the vial. If the whole plate was used, 500 μl beads were combined with 5.0 ml Diluent RD2-1. If fewer wells were used, the volumes were adjusted accordingly. The vial or wells were protected from light.
(iii) If necessary: sample preparation. All samples were used undiluted unless the sample concentration exceeded the highest standard value (S1). In such a case, the assay was repeated with an appropriately diluted sample. For the dilution PTT6 was used. All samples were measured in triplicates.
(iv) The beads were gently vortexed before 50 μl were added to each well using a multi-channel pipet and a reservoir.
(v) 50 μl of standard or sample were added per well. Then, the plate was covered with plate sealer and incubated protected from light for 2 h at room temperature (RT) on an orbital shaker at 800 rpm.
(vi) During sample incubation, the Biotin Antibody Cocktail was prepared by gently vortexing the vial to re-suspend. It was important to be careful not to invert the vial. Then 500 μl Biotin Antibody Cocktail was combined with 5.0 ml Diluent RD2-1. The solution was mixed thoroughly.
(vii) Streptavidin phycoerythrin (PE) was prepared by gently vortexing the vial to re-suspend (carefully not to invert the vial). Then, 200 μl Streptavidin-PE concentrate was combined with 5.35 ml Wash Buffer. The solution was mixed thoroughly and protected from light.
(Viii) The plate was washed as follows: The plate was attached to the magnet and let sit for at least a minute. While the plate was attached to the magnet, the plate was quickly inverted to decant the plate into the sink followed by a relatively forceful downward motion (1-2 times) to empty the wells. Complete removal of liquid by inversion was essential, but did not blot the plate. The magnet was detached and the wells were filled with 100 μl Wash Buffer using a multi-channel pipette. Afterwards, the magnet was re-attached and let sit for a minute before the decanting was performed as described before. The washing was repeated for a total number of 3 washed.
(ix) 50 μl diluted Biotin Antibody were added to each well using a multi-channel pipette. The plate was covered and incubated for 1 h at RT on shaker set at 800 rpm. Afterwards, the plate was washed 3 times as before.
(x) 50 μl diluted Streptavidin-PE were added to each well. The plate was covered and incubated for 30 min at RT on shaker as before. Afterwards, the plate was washed 3 times as before.
(xi) 100 μl Wash Buffer were added to each well and the plate was incubated for 2 min at RT on shaker as before. Then, the well contents were immediately transferred into a Costar 6509 96-well4 plate using a multi-channel pipette set to 120 μl. The plate was then placed into the fitting mold in the Luminex 3D scanner.
(xii) The plate was read and analyzed using Luminex 3D and Xponent software.

### TGF-β1 Singleplex:

(i) A standard was prepared by using 1.5 ml polypropylene tubes to make dilutions. For this purpose, 500 μl Standard S1 was pipetted into a S1 tube. To tubes S2-S6, 200 μl of complete PTT6 medium were added. By transferring 100 μl serially from one S1 up to S7 and ensuring a thoroughly mixing, the standard was diluted 1:3.
(ii) Beads were prepared by gently vortexing the vial to re-suspend. It was important to be careful not to invert the vial. If the whole plate was used, 50 μl beads were combined with 5.0 ml Microparticle Diluent RD2-1. If fewer wells were used, the volumes were adjusted accordingly. The vial or wells were protected from light.
(iii) To make TGF-β1 immunoreactive (samples only, not the standard), 30 μl of Activation Reagent was added to 150 μl supernatant. The solution was mixed thoroughly and incubated for 10 min at RT. All samples were used undiluted unless the sample concentration exceeded the highest standard value (S1). In such a case, the assay was repeated with an appropriately diluted sample. For the dilution PTT6 was used. All samples were measured in triplicates.
(iv) The beads were gently vortexed before 50 μl were added to each well using a multi-channel pipet and a reservoir.
(v) 50 μl of standard or sample were added per well. Then, the plate was covered with plate sealer and incubated protected from light for 2 h at RT on an orbital shaker at 800 rpm.
(vi) During sample incubation, the Biotin Antibody Cocktail was prepared by gently vortexing the vial to re-suspend. It was important to be careful not to invert the vial. Then 50 μl Biotin Antibody Concentrate was combined with 5.0 ml Biotin Antibody Diluent. The solution was mixed thoroughly.
(vii) Streptavidin phycoerythrin (PE) was prepared by gently vortexing the vial to re-suspend (carefully not to invert the vial). Then, 55 μl 100× Streptavidin-PE concentrate was combined with 5.35 ml Wash Buffer. The solution was mixed thoroughly and protected from light.
(viii) The plate was washed as follows: The plate was attached to the magnet and let sit for at least a minute. While the plate was attached to the magnet, the plate was quickly inverted to decant the plate into the sink followed by a relatively forceful downward motion (1-2 times) to empty the wells. Complete removal of liquid by inversion was essential, but did not blot the plate. The magnet was detached and the wells were filled with 100 μl Wash Buffer using a multi-channel pipette. Afterwards, the magnet was re-attached and let sit for a minute before the decanting was performed as described before. The washing was repeated for a total number of 3 washed.
(ix) 50 μl diluted Biotin Antibody were added to each well using a multi-channel pipette. The plate was covered and incubated for 1 h at RT on shaker set at 800 rpm. Afterwards, the plate was washed 3 times as before.
(x) 50 μl diluted Streptavidin-PE were added to each well. The plate was covered and incubated for 30 min at RT on shaker as before. Afterwards, the plate was washed 3 times as before.
(xi) 100 μl Wash Buffer were added to each well and the plate was incubated for 2 min at RT on shaker as before. Then, the well contents were immediately transferred into a Costar 6509 96-well4 plate using a multi-channel pipette set to 120 μl. The plate was then placed into the fitting mold in the Luminex 3D scanner.
(xii) The plate was read and analyzed using Luminex 3D and Xponent software.

### Stage 3: Master Cell Bank

3 cell lines that meet the Stage 2 in-process release criteria are then seeded into the Terumo Quantum bioreactor at 20-40×10⁶ viable cells and propagated for approximately 7-14 days. Post-Quantum cells are tested for sterility, endotoxin, mycoplasma, human pathogen virus and adventitious virus testing. The cells are cryopreserved at 9 to 10×10⁶ cells/vial (50-70 vials per batch). The MSC are propagated in flasks and in the Terumo Quantum in culture medium PTT6, formulated as follows for 1000 ml (500 ml of PTT6 basal medium, 236 ml of M171, 236 ml of DMEM F12, 25 ml of fetal bovine serum, 0.1 ml of 0.1 mg/ml epidermal growth factor [10 ng/ml final concentration]), 0.35 ml of insulin (5 μg/ml final concentration) and incubated at 37° C. in 5% CO2.

### Stage 4: Therapeutic Cultures

1×, 3× and 5×10⁶ MSCs that were positively tested for Ang-1, TGF-β, VEGF and HGF were bottled in Hypothermosol® and stored at 2-8° C. before pharmaceutical administration.

### Example 2: Protein Secretion Level Analysis for Identifying Suitable Donor Cords

For analysis, 10 umbilical cords were collected from different donors. These umbilical cords were used to produce 10 individual MSC populations derived from the amniotic membrane of the umbilical cord.

The aim of the experiment was to determine the secretion level of Ang-1, VEGF, HGF and TGF-β1 to draw back a conclusion about variabilities in the secretion profile of individual MSC populations and to identify a MSC population with sufficient secretion of Ang-1, VEGF, HGF and TGF-β1.

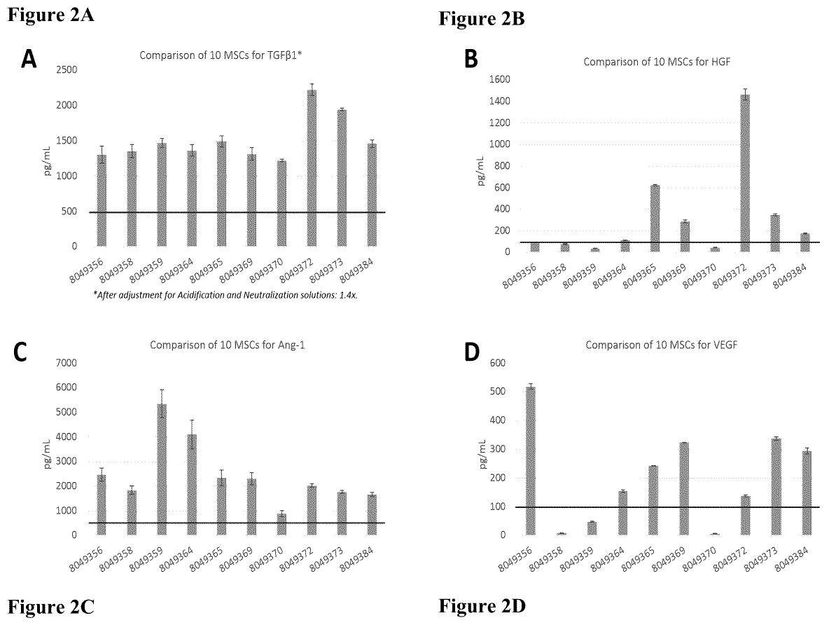

Therefore, the individual MSC populations were cultivated according to the present invention and post-stage 2 the secretion level of Ang-1, VEGF, HGF and TGF-β (here TGF-β1) was determined for each the MSC population as described in Example 1. The results of the secretion level analysis are shown in FIG. 2, wherein the protein specific threshold (500 μg/ml for Ang-1 and TGF-β, respectively, and 100 pg/m for VEGF and HGF, respectively) was indicated by a horizontal, black line.

As can be seen from in the result, the secretion level of TGF-β1 is more than twice as high as the threshold value in all 10 individual MSC populations. Thus, the threshold value of 500 pg/m for TGF-β1 was exceeded for all individual MSC populations. The plotted secretion levels of HGF show that 5 out of 10 samples exceed the threshold value of 100 pg/m, namely MSC population 8049365 exhibiting a secretion level of about 600 pg/m, MSC population 8049369 exhibiting a secretion level of about 300 pg/m, MSC population 8049372 exhibiting a secretion level of about 1450 pg/m, MSC population 8049373 exhibiting a secretion level of about 380 pg/m and MSC population 8049384 exhibiting a secretion level of about 190 pg/m. The plotted secretion levels Ang-1 show that all individual MSC populations exceeded the threshold value of 500 pg/m. The secretion levels of VEGF show that the threshold value of 100 pg/m was exceeded for all samples except 8049358, 8049359 and 8049370. In this context, the secretion level of 8049359 was only about 50 pg/m, whereas the levels of U.S. Pat. Nos. 8,049,358 and 8,049,359 are close to zero.

The results show that the protein secretion level of the different individual MSC populations varies, confirming that MSCs have individual secretion profiles. In order to identify a MSC population with sufficient secretion of Ang-1, VEGF, HGF and TGF-β1, the secretion level of these proteins was analyzed.

In this context, MSC exhibit sufficient secretion of Ang-1, TGF-β, VEGF and HGF if the levels of these proteins exceed their respective threshold value. Accordingly, MSC population 8049365, 8049369, 8049372, 8049373 and 8049384 (each of which is obtained from a different donor umbilical cord) exhibit sufficient Ang-1, TGF-β, VEGF and HGF secretion since these are the only MSC populations that exceed the given thresholds value for each of the four chosen proteins. For the subsequent experiments (setting up a master cell bank and production of cells for pharmaceutical purposes, the MSC population 8049372 was chosen herein.

### Example 3: Analysis of Secretion Level Stability

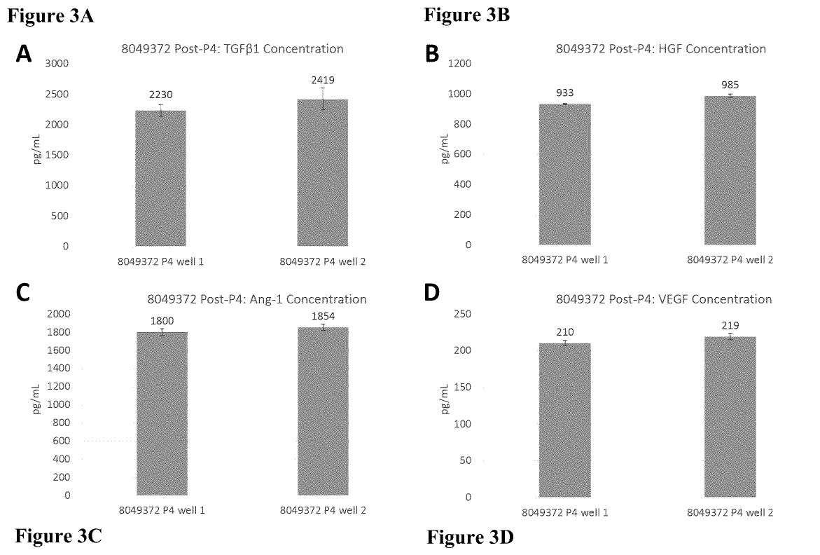

MSC population 8049372 showing sufficient secretion of the proteins Ang-1, TGF-β, VEGF and HGF in Example 2 was used to analyze the stability of the protein secretion level. Therefore, the MSC population was further cultivated until the cells were passaged a fourth time. Then, two samples of the MSC population post-stage 4 (MSC of well 1 and MSC of well 2) were analyzed regarding the secretion level of Ang-1, VEGF, HGF and TGF-β (here TGF-β1) as described in Example 1. The protein secretion levels of the MSC population 8049372 determined in this experiment (post-stage 4) is then compared to the secretion levels of the MSC population 8049372 determined in Example 2 (pots-stage 2). This way, changes in the secretion levels within different time points can be revealed. Thereby, conclusions can be drawn about the stability of the protein secretion level and thus about the sufficiency of the protein secretion over time. The results are shown in FIG. 3.

Post-stage 2, MSC population 8049372 exhibited a secretion level of about 2200 pg/m for TGF-β1. After passaging the cells two more times, the post-stage 4 MSC population 8049372 exhibits in average about 2345 pg/m TGF-β1. Thus, the secretion level of MSC population 8049372 increased about 7% after passaging the cells two more times. The secretion level of HGF decreased about 33% after passaging the cells two more times from about 1450 pg/m post-stage 2 to about 959 pg/m in average post-stage 4. The post-stage 4 MSC population 8049372 exhibits in average about 1827 pg/m Ang-1, which is a decrease of about 9% regarding the 2000 pg/m Ang-1 post-stage 2. The secretion level of VEGF increased about 43% after passaging the cells two more times from about 150 pg/m post-stage 2 to about 215 pg/m in average post-stage 4.

The results show, that the secretion level of the proteins Ang-1, VEGF, HGF and TGF-β (here TGF-β1) also varies after passaging the cells two more times. However, the respective thresholds values as chosen herein were still exceeded for all of the analyzed proteins after passaging the cells two more times. Consequently, the results of this experiment indicate that MSC population 8049372 maintained a relative protein secretion stability and thus its wound healing potency. Therefore, this results show that the wound healing potency of a MSC population may be stable for a certain period. Based on this result, the MSC population 8049372 is a suitable candidate as starting material for generating a master cell bank or for the production of a pharmaceutical composition for subsequent administration to a subject.

### Example 4: Identification of a Medium for Suitable for Inducing or Improving Wound Healing Properties of a Mesenchymal Stem Cell

For this experiment, various isolated MSC populations of the amniotic membrane of umbilical cord were cultivated in PTT4, PTT6 or DMEM/F12 as described in International Application WO 2018/067071 and subsequently analyzed with respect to the secretion of wound healing marker proteins for comparison.

### Culture Protocol for Cultivation of the Isolated MSCs

5 million MSCs were plated in 100 mm tissue culture dishes in DMEM/F12/10% FCS for 24 h.
Medium was discarded and PTT4, PTT6/DMEM/F12 was added to culture for 24 h.
Medium was discarded and cells were washed with PBS.
10 ml DMEM were added to culture for 24 h.
Medium was discarded and 5 ml DMEM were added to culture.
After 24 h cultivation, conditioned media were harvested, centrifuged to remove cell debris, supernatant aliquoted into tubes for storage at −80° C. and subsequent analysis of marker protein secretion by cytokine assays

Secretion Level Analysis on PTT4, PTT6 vs. DMEM/F12 Media Supernatants Secretion level analysis was performed in MSC supernatants. Measurements and analysis has been conducted using Luminex 200 and Xponent software.

Each sample was tested in triplicate except the samples of supernatant of placental The aim of this experiment was to generate cytokine profiles of MSCs cultivated either in PTT4 or PTT6 and to compare the profiles of MSCs from different tissue origins (umbilical cord lining vs. Wharton's Jelly vs. placental MSC). The cytokine measurements were carried as described below. The profile will shed light onto which stem cell population grown in which medium would secrete more of the cytokines of interest, meaning which medium is suitable for inducing or promoting wound healing properties of the MSCs.

### Multiplex Analysis

### Multiplex Information:

R&D Systems/Bio-techne cat. #LXSAHM. This kit is lot #L123680, expires Aug. 28, 2018, with the following analytes:
Ang-1, angiopoietin
VEGF, vascular endothelial growth factor.
HGF, hepatocyte growth factor

### TGFβ1 Singleplex Information: R&D Systems/Bio-Techne:

Base kit, cat. #LTGM00, lot #P156217, received Feb. 27, 2018, expires Aug. 30, 2018.
TGFβ1 component, cat. #LTGM100, lot #P161760, received Feb. 27, 2018, expires Nov. 27, 2019.

### Multiplex Information:

R&D Systems/Bio-techne cat. #LXSAHM. This kit is lot #L123999, expires Sep. 25, 2018, with the following analytes:
Ang-1, angiopoietin.
VEGF, vascular endothelial growth factor
HGF, hepatocyte growth factor

### Data Entry

Raw data output is in PDF and Excel formats. Data in Excel format are used to process the data.

### Procedure

Protein detection in MSC supernatants was carried out in accordance with the detailed protocol information. As part of this experiment, the protocol has a single amendment: Std. 8 in the Multiplex kit is no longer used. The reason for discontinuing Std. 8 is because R&D Systems protocol itself uses only Standards 1 through 6. Furthermore, Std. 8 was validated for only two of the six analytes that comprise the Multiplex: HGF. In the case of HGF, that analyte falls in the mid-region of the standard curve. Since the Standards are reconstituted using growth media, standard curves were constructed with both PTT4 and PTT6. Test samples grown in either PTT4 or PTT6 were extrapolated from respective standard curves. The results were extrapolated by the Luminex software from the analyte-specific standard curve that is generated by the same software: the analysis algorithm is set to Logistic 5P Weighted with weighted analysis, using 1/y2 for weighting.

### Samples

1. DMEM/F12 and PTT6 and PTT4 media (not exposed to MSCs)
2. Supernatants of MSC's to be tested
3. Optional: supernatants from different donors; CR001A, C, D, and G.

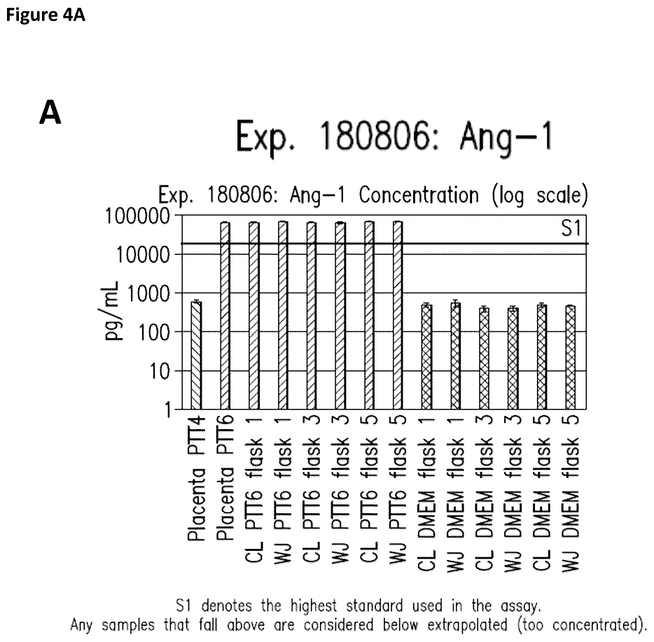

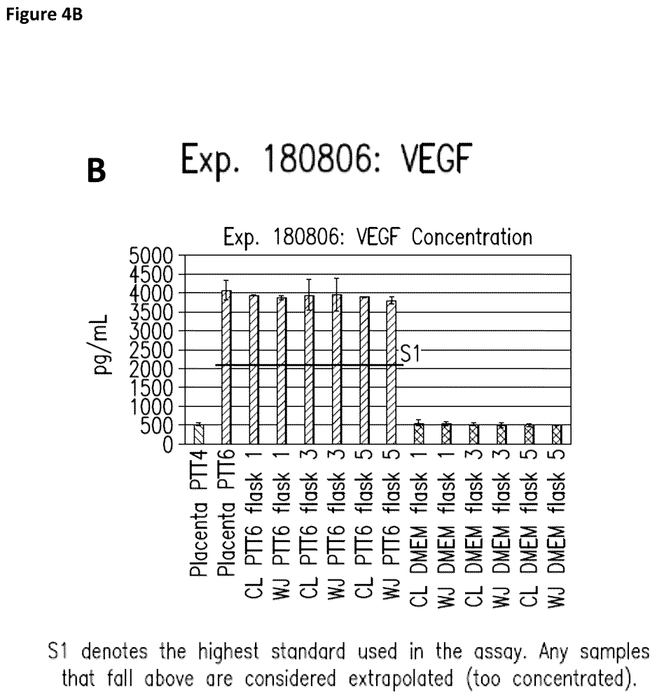

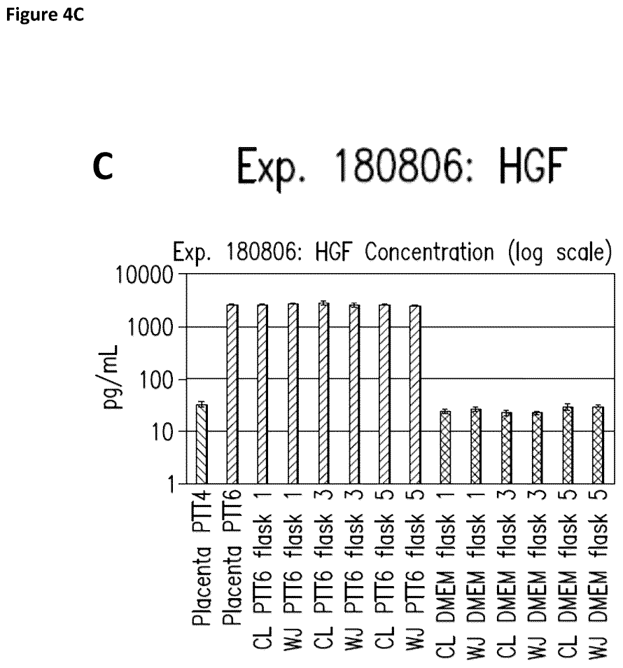

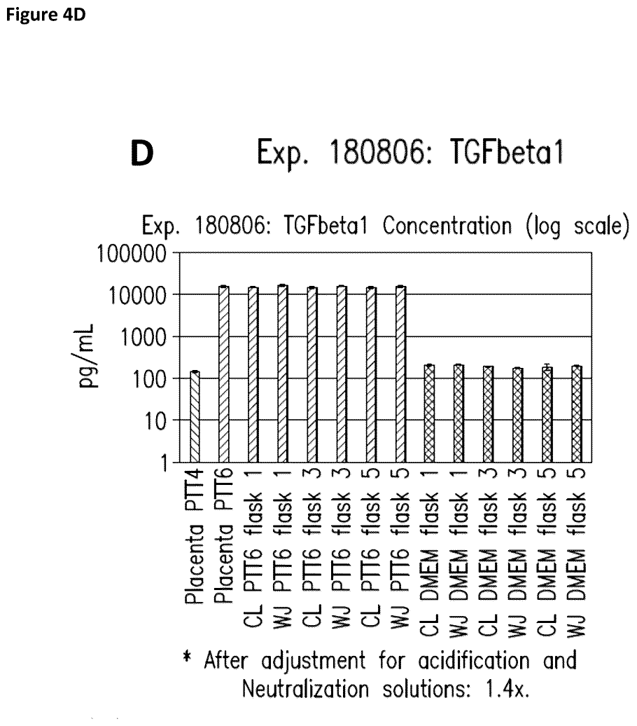

The results for Ang-1 are shown in FIG. 4A and show that MSC of the amniotic membrane of the umbilical cord produce more Ang-1 when grown in PTT6 than when grown in DMEM/F12 or PTT4. The results for VEGF are shown in FIG. 4B and show that MSC of the amniotic membrane of the umbilical cord produce more VEGF when grown in PTT6 than when grown in DMEM/F12 or PTT4. The results for HGF are shown in FIG. 4C and show that MSC of the amniotic membrane of the umbilical cord produce more HGF when grown in PTT6 than when grown in DMEM/F12 or PTT4. Finally, the results for TGF-β1 are shown in FIG. 4D and show that MSC of the amniotic membrane of the umbilical cord produce more TGF-β1 when grown in PTT6 than when grown in DMEM/F12 or PTT4.

From the above described experiments the following can be concluded. When MSCs are cultivated in PTT6 medium, the secretion of Ang-1, TGF-β1, VEGF, and HGF by the MSC population is significantly increased compared to their secretion level in PTT4 or a commercially available culture medium such as DMEM/F12. PTT6 medium has the highest ability to induce or improve wound healing properties of a MSC population by promoting the secretion of all of Ang-1, TGF-β1, VEGF, and HGF (the involvement of which in wound healing is known, as discussed herein) in MSC populations Therefore, determining the secretion level of Ang-1, TGF-β1, VEGF, and HGF can be used to identify a medium suitable for inducing or improving wound healing properties of a mesenchymal stem cell population.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention. The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group. Further embodiments of the invention will become apparent from the following claims.

The invention is further characterized by the following items:
1. A method of assessing the wound healing potency of a mesenchymal stem cell population, wherein the method comprises determining in a medium the level of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) secreted into the medium by the mesenchymal stem cell population.
2. A method of identifying a tissue suitable as starting material for producing a mesenchymal stem cell population for pharmaceutical use, wherein the method comprises determining in a medium the level of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) secreted into the medium by a sample of the tissue or a cell isolated from the tissue.
3. A method of selecting a mesenchymal stem cell population for producing a stem cell population under cGMP conditions, wherein the method comprises determining in a medium the level of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) secreted into the medium by the mesenchymal stem cell population.
4. A method of selecting a mesenchymal stem cell population for producing a stem cell population for subsequent pharmaceutical administration, wherein the method comprises determining in a medium the level of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) secreted into the medium by the mesenchymal stem cell population.
5. A method of selecting a mesenchymal stem cell population for generating a master cell bank, wherein the method comprises determining in a medium the level of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) secreted into the medium by the mesenchymal stem cell population.
6. The method of any one of the items 1 to 5, wherein the method comprises determining in the medium the level of at least two, at least three or all four proteins selected from the group consisting of Angiopoietin 1 (Ang-1), Transforming Growth Factor beta (TGF-β), Vascular Endothelial Growth Factor (VEGF) and Hepatocyte Growth Factor (HGF) secreted into the medium by the mesenchymal stem cell population.
7. The method of any one of the items 1 to 6, wherein a secretion level equaling or exceeding a threshold value indicates
   (i) potency for wound healing of the mesenchymal stem cell population; or
   (ii) a tissue or an isolated cell suitable as starting material for producing a mesenchymal stem cell population.
8. The method of item 7, wherein for Angiopoietin 1 (Ang-1) the threshold value is about 400 μg/ml or about 500 pg/m.
9. The method of items 7 or 8, wherein for Transforming Growth Factor beta (TGF-β) the threshold value is about 400 pg/m or about 500 pg/m.
10. The method of any one of the items 7 to 9, wherein for Vascular Endothelial Growth Factor (VEGF) the threshold value is about 80 mg/ml or about 100 pg/m.
11. The method of any one of the items 7 to 10, wherein for Hepatocyte Growth Factor (HGF) the threshold value is about 80 pg/m or about 100 pg/m.
12 The method of any of items 8 to 11, wherein the secretion level of all four proteins equal or exceed their respective threshold value, and wherein the threshold value is
   a threshold value of about 400 pg/m for Angiopoietin 1 (Ang-1),
   a threshold value of about 400 pg/m for Transforming Growth Factor beta (TGF-β),
   a threshold value of about 80 pg/m for Vascular Endothelial Growth Factor (VEGF), and
   a threshold value of about 80 pg/m for Hepatocyte Growth Factor (HGF).
13. The method of any of items 8 to 11, wherein the secretion level of all four proteins equal or exceed their respective threshold value, and wherein the threshold value is
   a threshold value of about 500 pg/m for Angiopoietin 1 (Ang-1),
   a threshold value of about 500 pg/m for Transforming Growth Factor beta (TGF-β),
   a threshold value of about 100 pg/m for Vascular Endothelial Growth Factor (VEGF), and
   a threshold value of about 100 pg/m for Hepatocyte Growth Factor (HGF).
14. The method of any one of the items 1 to 13, wherein the mesenchymal stem cell population is selected from the group consisting of a mesenchymal stem cell population of the umbilical cord, a placental mesenchymal stem cell population, a mesenchymal stem cell population of the cord-placenta junction, a mesenchymal stem cell population of the cord blood, a mesenchymal stem cell population of the bone marrow, and an adipose-tissue derived mesenchymal stem cell population.
15. The method of item 14, wherein the mesenchymal stem cell population of the umbilical cord is selected from the group consisting of a mesenchymal stem cell population of the amnion (AM), a perivascular (PV) mesenchymal stem cell population, a mesenchymal stem cell population of Wharton's jelly (WJ), a mesenchymal stem cell population of the amniotic membrane of umbilical cord and a mixed mesenchymal stem cell population of the umbilical cord (MC).
16. The method of item 2, wherein the tissue is an umbilical cord or the amniotic membrane of the umbilical cord and the mesenchymal stem cell population is a stem cell population of the amniotic membrane of umbilical cord.
17. The method of items 15 or 16, wherein the mesenchymal stem cell population of the amniotic membrane of the umbilical cord is a mesenchymal stem cell population, wherein at least about 90% or more cells of the stem cell population express each of the following markers: CD73, CD90 and CD105.
18. The method of item 17, wherein at least about 90% or more cells of the mesenchymal stem cell population lack expression of the following markers: CD34, CD45 and HLA-DR.
19. The method of items 17 or 18, wherein at least about 91% or more, about 92% or more, about 93% or more, about 94% or more, about 95% or more, about 96% or more, about 97% or more, about 98% or more about 99% or more cells of the mesenchymal stem cell population express each of CD73, CD90 and CD105 and lack expression of each of CD34, CD45 and HLA-DR.
20. The method of any of the foregoing items, wherein the medium is a cell culture medium or a storage medium.
21. The method of item 20, wherein the storage medium is Hypothermosol or Plasmalyte.
22. The method of any one of the items 1 to 19, wherein the medium comprises Dulbecco's Modified Eagle's Medium (DMEM) in a final concentration of about 55 to 65% (v/v), Ham's F12 Medium (F12) in a final concentration of about 5 to 15% (v/v), a serum free basal medium in a final concentration of about 15 to 30% (v/v) and Fetal Bovine Serum (FBS) in a final concentration of about 1 to 8% (v/v).
23. The method of item 22, wherein the medium comprises Dulbecco's Modified Eagle's Medium (DMEM) in a final concentration of about 57.5 to 62.5% (v/v), Ham's F12 Medium (F12) in a final concentration of about 7.5 to 12.5% (v/v), a serum free basal medium in a final concentration of about 17.5 to 25.0% (v/v) and Fetal Bovine Serum (FBS) in a final concentration of about 1.75 to 3.5% (v/v).
24. The method of item 23, wherein the medium comprises Dulbecco's Modified Eagle's Medium (DMEM) in a final concentration of about 61.8% (v/v), Ham's F12 Medium (F12) in a final concentration of about 11.8% (v/v), a serum free basal medium in a final concentration of about 23.6% (v/v) and Fetal Bovine Serum (FBS) in a final concentration of about 2.5% (v/v).
25. The method of items 23 or 24, wherein the serum free basal medium is M171.
26. The method of any of items 22 to 25, wherein the medium further comprises Epidermal Growth Factor (EGF) in a final concentration of about 1 ng/ml to about 20 ng/ml.
27. The method of item 26, wherein the medium comprises Epidermal Growth Factor (EGF) in a final concentration of about 10 ng/ml.
28. The method of any of items 22 to 27, wherein the medium comprises Insulin in a final concentration of about 1 pg/m to 10 pg/m.
29. The method of item 28, wherein the medium comprises Insulin in a final concentration of about 5 pg/m.
30. The method of any of items 22 to 29, wherein the medium further comprises at least one of the following supplements: adenine, hydrocortisone, and 3,3′,5-Triiodo-L-thyronine sodium salt (T3).
31. The method of any of items 22 to 30, wherein the medium comprises all three of adenine, hydrocortisone, and 3,3′,5-Triiodo-L-thyronine sodium salt (T3).
32. The method of item 30 or 31, wherein the medium comprises adenine in a final concentration of about 0.01 to about 0.1 pg/m adenine, hydrocortisone in a final concentration of about 0.1 to about 10 pg/m hydrocortisone and/or 3, 3′, 5-Triiodo-L-thyronine sodium salt (T3) in a final concentration of about 0.5 to about 5 ng/ml.
33. The method of any of items 1 to 32, wherein cultivating the mesenchymal stem cell population in the medium as defined in any of the foregoing items 22 to 32 results in an increase of the expression and/or secretion of at least one of the proteins selected from a group consisting of Angiopoietin 1 (Ang-1), Transforming Growth Factor beta (TGF-β; in particular TGF-β1), Vascular Endothelial Growth Factor (VEGF) and Hepatocyte Growth Factor (HGF) by the mesenchymal stem cell population relative to a reference medium that does not comprise all of DMEM (Dulbecco's modified eagle medium), F12 (Ham's F12 Medium), M171 (Medium 171) and FBS (Fetal Bovine Serum).
34. The method of any one of the items 1 to 33, wherein the cell medium is subjected to centrifugation after a suitable period of cultivation.
35. The method of item 34, wherein the suitable period of cultivation comprises about 12 h, about 24 h, about 36 h, about 46 h, about 48 h or about 50 h, preferably about 48 h.
36. The method of items 34 or 35, wherein the supernatant of the centrifuged cell culture medium is subjected to a multiplex assay.
37. The method of item 36, wherein the multiplex assay is bead-based.
38. Use of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) for assessing the wound healing potency of a mesenchymal stem cell population.
39. Use of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) for selecting a mesenchymal stem cell population for producing a stem cell population under cGMP conditions.
40. Use of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) for selecting a mesenchymal stem cell population for producing a stem cell population for subsequent pharmaceutical administration.
41. Use of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) for selecting a mesenchymal stem cell population for generating a master cell bank.
42. Use of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) for identifying a tissue suitable as starting material for producing a mesenchymal stem cell population for pharmaceutical use.
43. The use of item 42, wherein the tissue is an umbilical cord or the amniotic membrane of the umbilical cord and the mesenchymal stem cell population is a stem cell population of the amniotic membrane of umbilical cord.
44. The use of any of items 38 to 41, wherein the use comprises determining in the cell culture medium the level of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) secreted into the cell culture medium by the mesenchymal stem cell population.
45. The use of item 42 or 43, wherein the use comprises determining in the cell culture medium the level of at least one protein selected from the group consisting of Angiopoietin 1 (Ang 1), Transforming Growth Factor beta (TGF B), Vascular Endothelial Growth Factor (VEGF) and Hepatocyte Growth Factor (HGF) secreted into the cell culture by a sample of the tissue or a cell isolated from the tissue.
46. The use of any of items 38 to 44 wherein the use comprises determining in the cell culture medium the level of at least two, at least three or all four proteins selected from the group consisting of Angiopoietin 1 (Ang-1), Transforming Growth Factor beta (TGF-β), Vascular Endothelial Growth Factor (VEGF) and Hepatocyte Growth Factor (HGF) secreted into the cell culture medium by the mesenchymal stem cell population.
47. A method of identifying a medium suitable for inducing or improving wound healing properties of a mesenchymal stem cell population, wherein the method comprises determining the level of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) secreted into the medium by the mesenchymal stem cell population.
48. Use of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) for identifying a medium suitable for inducing or improving wound healing properties of a mesenchymal stem cell population.
49. The use of item 48, wherein the use comprises determining in the medium the level of at least two, at least three or all four proteins secreted into the medium by the mesenchymal stem cell population.

What is claimed is:

1. A method of identifying a tissue suitable as starting material for producing a mesenchymal stem cell population for pharmaceutical use, wherein the method comprises determining in a medium the level of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) secreted into the medium by a sample of the tissue or a cell isolated from the tissue.

2. A method of selecting a mesenchymal stem cell population for producing a stem cell population under cGMP conditions, wherein the method comprises determining in a medium the level of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) secreted into the medium by the mesenchymal stem cell population.

3. A method of selecting a mesenchymal stem cell population for producing a stem cell population for subsequent pharmaceutical administration, wherein the method comprises determining in a medium the level of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) secreted into the medium by the mesenchymal stem cell population.

4. A method of selecting a mesenchymal stem cell population for generating a master cell bank, wherein the method comprises determining in a medium the level of at least one protein selected from the group consisting of Angiopoietin 1 (Ang-1), Transforming Growth Factor beta (TGF-β), Vascular Endothelial Growth Factor (VEGF) and Hepatocyte Growth Factor (HGF) secreted into the medium by the mesenchymal stem cell population.

5. The method of claim 1, wherein the method comprises determining in the medium the level of at least two, at least three or all four proteins selected from the group consisting of Angiopoietin 1 (Ang-1), Transforming Growth Factor beta (TGF-β), Vascular Endothelial Growth Factor (VEGF) and Hepatocyte Growth Factor (HGF) secreted into the medium by the mesenchymal stem cell population.

6. The method of claim 1, wherein a secretion level equaling or exceeding a threshold value indicates
(i) a tissue or an isolated cell suitable as starting material for producing a mesenchymal stem cell population.

7. The method of claim 6, wherein for Angiopoietin 1 (Ang-1) the threshold value is about 400 pg/m or about 500 pg/m.

8. The method of claim 6, wherein for Transforming Growth Factor beta (TGF-β) the threshold value is about 400 pg/m or about 500 pg/m.

9. The method of claim 6, wherein for Vascular Endothelial Growth Factor (VEGF) the threshold value is about 80 mg/ml or about 100 pg/m.

10. The method of claim 6, wherein for Hepatocyte Growth Factor (HGF) the threshold value is about 80 pg/m or about 100 pg/m.

11. The method of claim 6, wherein the secretion level of all four proteins equal or exceed their respective threshold value, and wherein the threshold value is
a threshold value of about 400 pg/m for Angiopoietin 1 (Ang-1),
a threshold value of about 400 pg/m for Transforming Growth Factor beta (TGF-β),
a threshold value of about 80 pg/m for Vascular Endothelial Growth Factor (VEGF), and
a threshold value of about 80 pg/m for Hepatocyte Growth Factor (HGF).

12. The method of claim 6, wherein the secretion level of all four proteins equal or exceed their respective threshold value, and wherein the threshold value is
a threshold value of about 500 pg/m for Angiopoietin 1 (Ang-1),
a threshold value of about 500 pg/m for Transforming Growth Factor beta (TGF-β),
a threshold value of about 100 pg/m for Vascular Endothelial Growth Factor (VEGF), and
a threshold value of about 100 pg/m for Hepatocyte Growth Factor (HGF).

13. The method of claim 1, wherein the mesenchymal stem cell population is selected from the group consisting of a mesenchymal stem cell population of the umbilical cord, a placental mesenchymal stem cell population, a mesenchymal stem cell population of the cord-placenta junction, a mesenchymal stem cell population of the cord blood, a mesenchymal stem cell population of the bone marrow, and an adipose-tissue derived mesenchymal stem cell population.

14. The method of claim 14, wherein the mesenchymal stem cell population of the umbilical cord is selected from the group consisting of a mesenchymal stem cell population of the amnion (AM), a perivascular (PV) mesenchymal stem cell population, a mesenchymal stem cell population of Wharton's jelly (WJ), a mesenchymal stem cell population of the amniotic membrane of umbilical cord and a mixed mesenchymal stem cell population of the umbilical cord (MC).

15. The method of claim 1, wherein the tissue is an umbilical cord or the amniotic membrane of the umbilical cord and the mesenchymal stem cell population is a stem cell population of the amniotic membrane of umbilical cord.

16. The method of claim 14, wherein the mesenchymal stem cell population of the amniotic membrane of the umbilical cord is a mesenchymal stem cell population, wherein at least about 90% or more cells of the stem cell population express each of the following markers: CD73, CD90 and CD105.

17. The method of claim 16, wherein at least about 90% or more cells of the mesenchymal stem cell population lack expression of the following markers: CD34, CD45 and HLA-DR.

18. The method of claim 16, wherein at least about 91% or more, about 92% or more, about 93% or more, about 94% or more, about 95% or more, about 96% or more, about 97% or more, about 98% or more about 99% or more cells of the mesenchymal stem cell population express each of CD73, CD90 and CD105 and lack expression of each of CD34, CD45 and HLA-DR.

19. The method of claim 1, wherein the medium is a cell culture medium or a storage medium.
